(19) United States
(12) Patent Application Publication
(10) Pub. No.: US 2015/0313632 A1
(43) Pub. Date: Nov. 5, 2015

(54) OBTURATOR WITH INSTRUMENT RETENTION

(71) Applicant: COVIDIEN LP, Mansfield, MA (US)

(72) Inventors: Gennady Kleyman, Brooklyn, NY (US); Gregory Fischvogt, Denver, CO (US); Gregory Okoniewski, North Haven, CT (US)

(21) Appl. No.: 14/651,347

(22) PCT Filed: Jan. 15, 2014
(86) PCT No.: PCT/US2014/011592

§ 371 Date: Jun. 11, 2015

Related U.S. Application Data
(60) Provisional application No. 61/754,014, filed on Jan. 18, 2013.
(60) Provisional application No. 61/754,022, filed on Jan. 18, 2013.
(60) Provisional application No. 61/754,026, filed on Jan. 18, 2013.

(51) Int. Cl. A61B 17/34; A61B 17/02; A61M 39/02

(52) U.S. Cl. CPC A61B 17/3417; A61M 39/0247; A61B 17/0218; A61B 2017/347; A61B 2017/3454; A61M 2039/027; A61M 2039/0279; A61M 2039/0294

(57) ABSTRACT

A surgical device having an obturator that includes a sidewall having an arcuate outer surface is disclosed. The sidewall may have an arcuate inner surface that defines a central longitudinal channel into which an instrument may be inserted. The sidewall may also define at least one bore extending between the arcuate inner and outer surfaces of the sidewall. An instrument retention element engages the arcuate outer surface of the obturator such that the instrument retention element extends radially into the bore. At least a portion of the instrument retention element may extend radially inward of the arcuate inner surface so as to engage, and restrict relative movement of, an instrument inserted into the central longitudinal channel of the obturator.

### CROSS-REFERENCE TO RELATED APPLICATIONS

This application is a National Stage Application of PCT/US2014/011592 under 35 USC §371 (a), filed Jan. 15, 2014, which claims the benefit of and priority to U.S. Provisional Application Ser. No. 61/754,014, filed Jan. 18, 2013, and U.S. Provisional Application Ser. No. 61/754,022, filed Jan. 18, 2013, and U.S. Provisional Application Ser. No. 61/754,026, filed Jan. 18, 2013, the disclosure of each is hereby incorporated by reference in its entirety.

### BACKGROUND

1. Technical Field

The present disclosure relates to an apparatus for penetration of body tissue. More particularly, the present disclosure relates to an obturator including an instrument holding mechanism for selectively securing an endoscope, laparoscope, or other surgical instruments within the obturator to provide visualization during penetration of the peritoneum or other body tissue.

2. Background of Related Art

In endoscopic surgical procedures, surgery is performed in any hollow viscus of the body through a small incision or through narrow endoscopic tubes (cannulas) inserted through a small entrance wound in the skin or through a naturally occurring orifice. In laparoscopic procedures, surgery is performed in the interior of the abdomen. Laparoscopic and endoscopic procedures often require the clinician to act on organs, tissues and vessels far removed from the incision, thereby requiring that any instruments used in such procedures be of sufficient size and length to permit remote operation. Generally, a trocar includes a stylet or obturator having a sharp tip for penetrating the body cavity. Typically, a trocar is used to puncture the body cavity and may include a cannula that remains in place for use during the laparoscopic procedure. Subsequently, the surgical region is insufflated thereby creating a working space.

It may be advantageous to provide an obturator that can be placed within an incision or body opening of a patient with a structure to retain a viewing instrument in a desired position during a surgical procedure.

### SUMMARY

Accordingly, the present disclosure relates to devices for accessing/penetrating body tissue during a surgical procedure. The present disclosure is directed to a surgical device for directly observing the penetration of the peritoneum or other body portions. The surgical device includes an obturator and an instrument retention element. The obturator includes a transparent window for passing optical images to a clinician, directly through an eyepiece or to an imaging system of an endoscope or laparoscope positioned within the obturator.

The device includes an elongate tubular member including a proximal end, a distal end, a sidewall defining a bore between the proximal and distal ends, and an instrument retention element that partially obstructs the bore. The device may include a housing near the proximal end. The instrument retention element is configured to receive an instrument. The instrument retention element may also be configured to hold instruments with a plurality of diameters. The instrument retention element may also be configured to restrict the movement of the instrument. The instrument may be a scope.

In an embodiment of the device, the instrument retention element is located between the housing and the distal end. In this embodiment, the instrument retention element is made from an elastomeric material. The elastomeric material may form an o-ring. The elastomeric material may expand around the instrument when receiving the instrument such that the interference between the instrument retention element and the instrument restricts the movement of the instrument. An inner surface of the sidewall may include a retainer to retain the instrument retention element in a longitudinal position within the elongate tubular body. The retainer may be a depression in the inner surface. The depression may form an annular groove. The retainer may also be parallel ribs protruding from the inner surface. The parallel ribs may form rings.

In another embodiment of the device, the instrument retention element may be located at the proximal end of the device. In this embodiment, the instrument retention element includes a flange at least partially obstructing the bore and at least one longitudinal slot through the instrument retention element. The slot may be configured to expand when the instrument retention element receives an instrument. The instrument retention element may be constructed of a rigid material.

In an embodiment, the device includes an elongate tubular member including a proximal end, a distal end, a sidewall defining a bore between the proximal and distal ends, and an instrument retention element which partially obstructs the bore. The elongate tubular member includes first and second flanges near the proximal end. The first and second flanges may be cylindrical. The first and second flanges define a housing which may include a slot configured to retain the instrument retention element. The instrument retention element is configured engage an instrument. The instrument retention element may be configured to engage instruments with a plurality of diameters. The instrument retention element may also be configured to restrict the movement of the instrument. The instrument may be a scope.

In a particular embodiment of the device, the instrument retention element is made from a compliant material located in the housing. In this embodiment, the instrument retention element may partially deform when the instrument retention element receives an instrument.

In another embodiment of the device, the instrument retention element is made from a rigid material. In this embodiment, the instrument retention element may include a backspan connecting two legs. The instrument retention element may act like a leaf spring when the instrument retention element engages an instrument. Each of the two legs may also include a locking mechanism. The locking mechanism may be a tab configured to be received by the housing. The tab may be configured to act like a clip to engage a wall of the housing.

The present disclosure relates to devices for accessing/penetrating body tissue during a surgical procedure. The present disclosure is directed to a surgical device for directly observing the penetration of the peritoneum or other body portions. The obturator may include a transparent window for passing optical images to a clinician, directly through an eyepiece or to an imaging system of an endoscope or laparoscope positioned within the obturator.

The device may include an elongate tubular member including a proximal end, a distal end, a sidewall defining a bore between the proximal and distal ends, and an instrument retention element which partially obstructs the bore. The instrument retention element may be configured to receive an instrument. The instrument retention element may also be configured to hold instruments with a plurality of diameters. The instrument retention element may also be configured to restrict the movement of the instrument. The instrument may be a scope.

In an embodiment of the device, the instrument retention element may be near the distal end of the elongate tubular member. The instrument retention element may be made of a compliant material which is configured to partially deform when receiving an instrument. In this embodiment, the instrument retention element may be positioned within a tip positioned at the distal end of the obturator. The instrument retention element may be secured to an interior surface of the sidewall or located in a recess of the interior surface.

In another embodiment of the device, the instrument retention element may be located between the housing and the distal end. In this embodiment, the instrument retention element may be made from an elastomeric material. The elastomeric material may form an o-ring. The elastomeric material may expand around the instrument when receiving the instrument such that the interference between the instrument retention element and the instrument restricts the movement of the instrument. An inner surface of the sidewall may include a retainer to retain the instrument retention element in a longitudinal position within the elongate tubular body. The retainer may be a depression in the inner surface. The depression may form an annular groove. The retainer may also be parallel ribs protruding from the inner surface. The parallel ribs may form rings.

In accordance with various embodiments, the present disclosure may also relate to a surgical device including an obturator and an instrument retention element. The obturator includes a sidewall having an arcuate inner surface that defines a central longitudinal channel into which an instrument may be inserted. The instrument retention element engages the sidewall of the obturator such that at least a portion of the o-ring extends radially inward of the arcuate inner surface so as to engage an instrument inserted into the central longitudinal channel of the obturator.

The instrument retention element may be configured to engage an endoscope. The instrument retention element may be configured to engage instruments of varying diameters. The instrument retention element may be configured to restrict the longitudinal movement of the instrument relative to the obturator. The instrument retention element may be made from an elastomeric material. The instrument retention element may be an o-ring.

In an embodiment, the arcuate inner surface has a retainer configured to retain the instrument retention element. The retainer may include at least one of a depression, an annular groove, a rib, or a ring. The obturator may include an elongate tubular member which forms a portion of the sidewall, the instrument retention element being located on the elongate tubular member. The obturator may include a handle which forms a portion of the sidewall, the instrument retention element being located on the handle.

In another embodiment, the present disclosure may relate to a surgical device, including an obturator and an instrument retention element. The obturator includes a sidewall having an arcuate outer surface. The sidewall has an arcuate inner surface that defines a central longitudinal channel into which an instrument may be inserted. The sidewall further defines at least one bore extending between the arcuate inner and outer surfaces of the sidewall. The instrument retention element engages the arcuate outer surface of the obturator such that the instrument retention element extends radially into the bore. At least a portion of the instrument retention element extends radially inward of the arcuate inner surface so as to engage an instrument inserted into the central longitudinal channel of the obturator.

The instrument retention element may be configured to engage an endoscope. The instrument retention element maybe configured to engage instruments having varying diameters. The instrument retention element may be further configured to restrict the longitudinal movement of the instrument relative to the obturator. The instrument retention element may be made from an elastomeric material. The instrument retention element may be an o-ring. The obturator may include an elongate tubular member which forms a portion of the sidewall, the instrument retention element being located on the elongate tubular member. The obturator may include a handle which forms a portion of the sidewall, the instrument retention element being located on the handle.

A method for using an obturator with any one of the presently disclosed instrument retention elements is also disclosed. The method includes making a first incision or accessing a naturally occurring orifice. A cannula is placed within the incision or orifice and secured in a desired position. The cannula is secured in the desired position and an obturator including one of the presently disclosed instrument retention elements is inserted into the cannula. A housing on the obturator may be configured to secure the obturator to the cannula. An instrument is inserted into a bore of the obturator and is received by the instrument retention element. The instrument retention element may be configured to allow the clinician to insert the instrument into a desired position within the bore and maintain the instrument in the desired position after the instrument is released by the clinician. The instrument may allow the clinician to directly or indirectly view a body cavity through the obturator.

In still another embodiment, the present disclosure may relate to a method of using a surgical device including providing an obturator, inserting an instrument into a central longitudinal channel of the obturator such that an instrument retention element engages the instrument and restricts longitudinal movement of the instrument relative to the obturator, and using the obturator to engage tissue during a surgical procedure. The obturator includes a sidewall having an arcuate outer surface. The sidewall has an arcuate inner surface that defines the central longitudinal channel. The sidewall further defines at least one bore extending between the arcuate inner and outer surfaces of the sidewall. The obturator also includes the instrument retention element engaging the arcuate outer surface of the obturator such that the instrument retention element extends radially into the bore. At least a portion of the instrument retention element extends radially inward of the arcuate inner surface. The obturator may be formed of a clear material. The instrument may be an endoscope. In such an arrangement, the obturator may be used to penetrate tissue while the endoscope provides a visual image of the tissue being penetrated.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a perspective view of a surgical device in accordance with the principles of the present disclosure illustrating an embodiment of an instrument retention element;

FIG. 2 is an enlarged view of the area of detail 2 of FIG. 1;

FIG. 3 is a top perspective view of the surgical device of FIG. 1 showing the instrument retention element removed from a slot in the housing;

FIG. 4 is a top perspective view of the surgical device of FIG. 1 showing the instrument retention element retained in a slot in the housing;

FIG. 5 is a side view of an embodiment of the instrument retention element;

FIG. 6 is a perspective view of the surgical device of FIG. 1 and a cannula;

FIG. 7 is a perspective view of the surgical device of FIG. 6 inserted into a cannula accessing a body cavity;

FIG. 8 is a side cross-sectional view taken along the line 8-8 in FIG. 7;

FIG. 9 is a top cross-sectional view taken along the line 9-9 in FIG. 8;

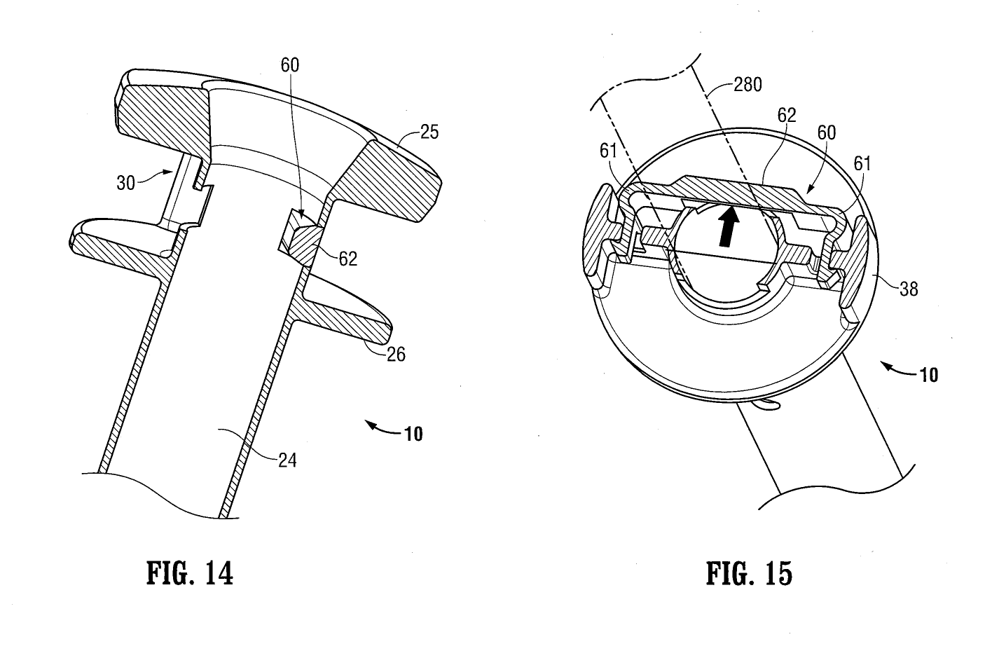

FIG. 10 is the side cross-sectional view of FIG. 8 with an instrument inserted in the bore;

FIG. 11 is a top cross-sectional view taken along the line 11-11 in FIG. 10;

FIG. 12 is a side perspective view of a surgical device in accordance with the principles of the present disclosure illustrating another embodiment of an instrument retention element;

FIG. 13 is a top perspective view of the cross-section taken along the line 13-13 in

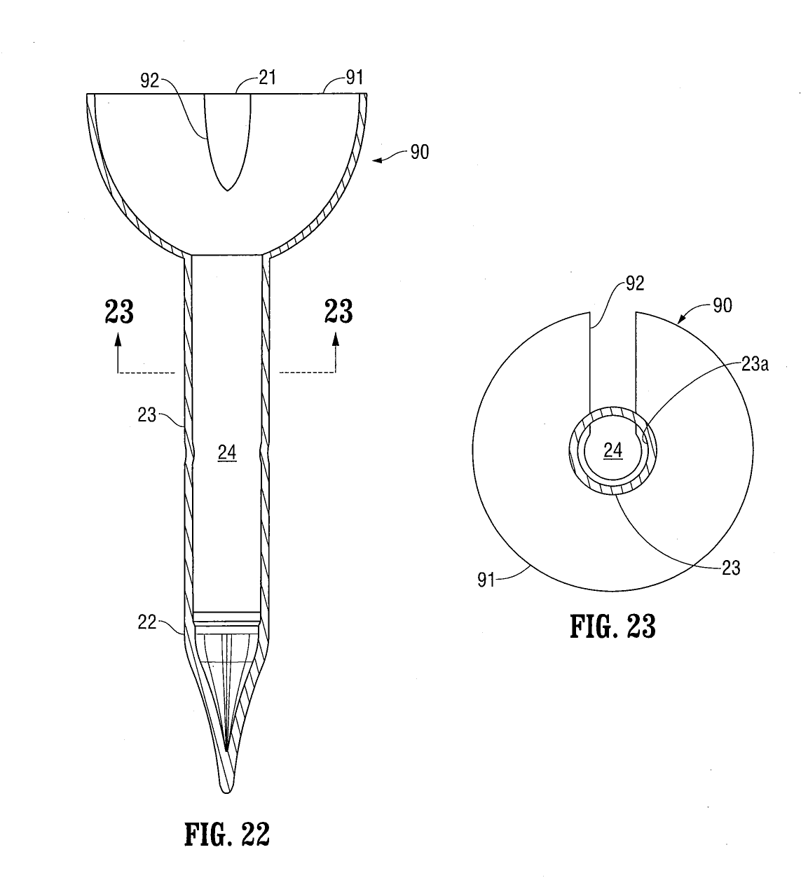

FIG. 12 illustrating the instrument retention element of the embodiment shown in FIG. 12;

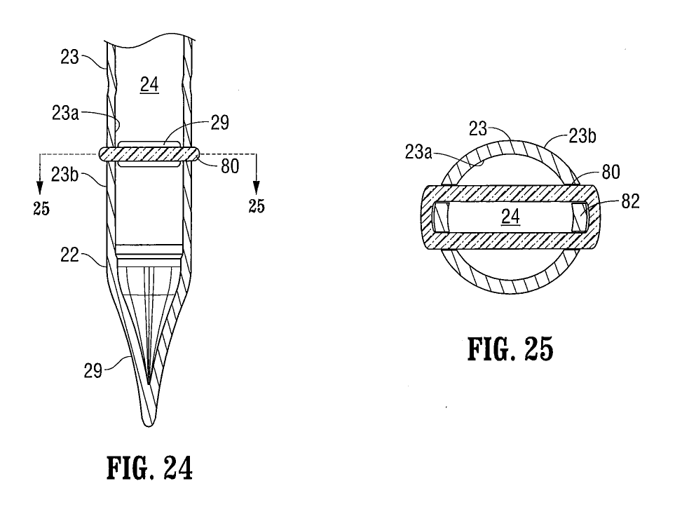

FIG. 14 is a side perspective view of the cross-section taken along the line 14-14 in FIG. 13;

FIG. 15 is the top perspective view of FIG. 14 with an instrument inserted showing the instrument retention element conforming to the diameter of the instrument;

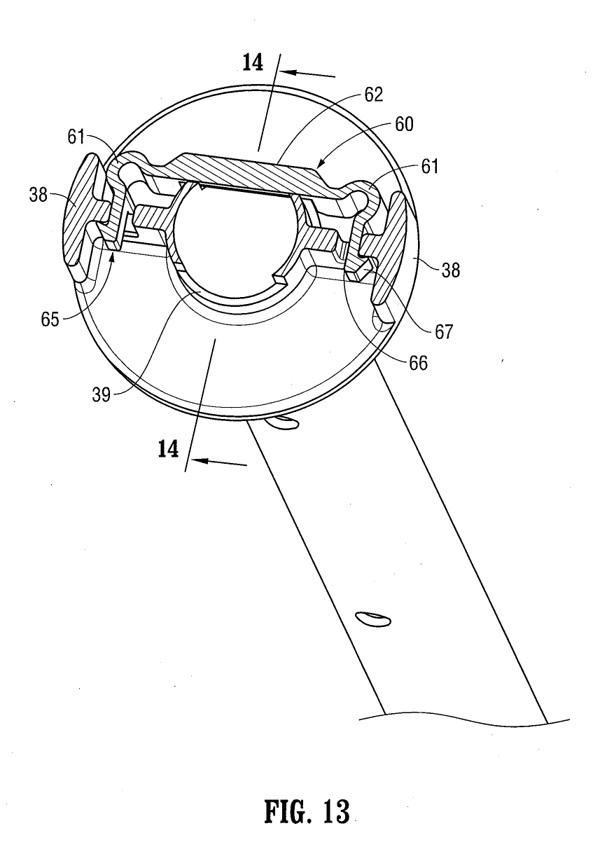

FIG. 16 is a side cross-sectional view similar to FIG. 8 showing an embodiment of a device consistent with the present disclosure with an instrument retention element located near the distal end;

FIG. 17 is a side cross-sectional view of the device shown in FIG. 16 the sidewall including a recess to retain the instrument retention element and with an instrument inserted in the bore including a structure configured to engage the instrument retention element;

FIG. 18 is a side cross-sectional view of the device shown in FIG. 16 with the instrument retention element in the tip;

FIG. 19 is a side cross-sectional view similar to FIG. 8 showing an embodiment of a device consistent with the present disclosure with an elastomeric instrument retention element located between the proximal and distal ends retained by a depression in the sidewall;

FIG. 20 is a top cross-sectional view taken along line 20-20 in FIG. 19;

FIG. 21 is a side cross-sectional view similar to FIG. 19 showing the instrument retention element retained by a plurality of parallel ribs;

FIG. 22 is a side cross-sectional view similar to FIG. 8 showing an embodiment of a device consistent with the present disclosure with an instrument retention element located at the proximal end including a slot;

FIG. 23 is a top cross-sectional view taken along line 23-23 in FIG. 22;

FIG. 24 is a side cross-sectional view similar to FIG. 19 showing an embodiment of a device consistent with the present disclosure with an elastomeric instrument retention element located between the proximal and distal ends supported by the outer surface of the obturator; and

FIG. 25 is a top cross-sectional view taken along line 25-25 in FIG. 24.

### DETAILED DESCRIPTION OF THE DRAWINGS

As used herein, the term “distal” refers to the portion that is being described which is farther from a user, while the term “proximal” refers to the portion that is being described which is closer to a user. In the following description, well-known functions or constructions are not described in detail to avoid obscuring the present disclosure in unnecessary detail. Further, to the extent consistent, any of the embodiments described herein may be used in conjunction with any or all of the other embodiments described herein.

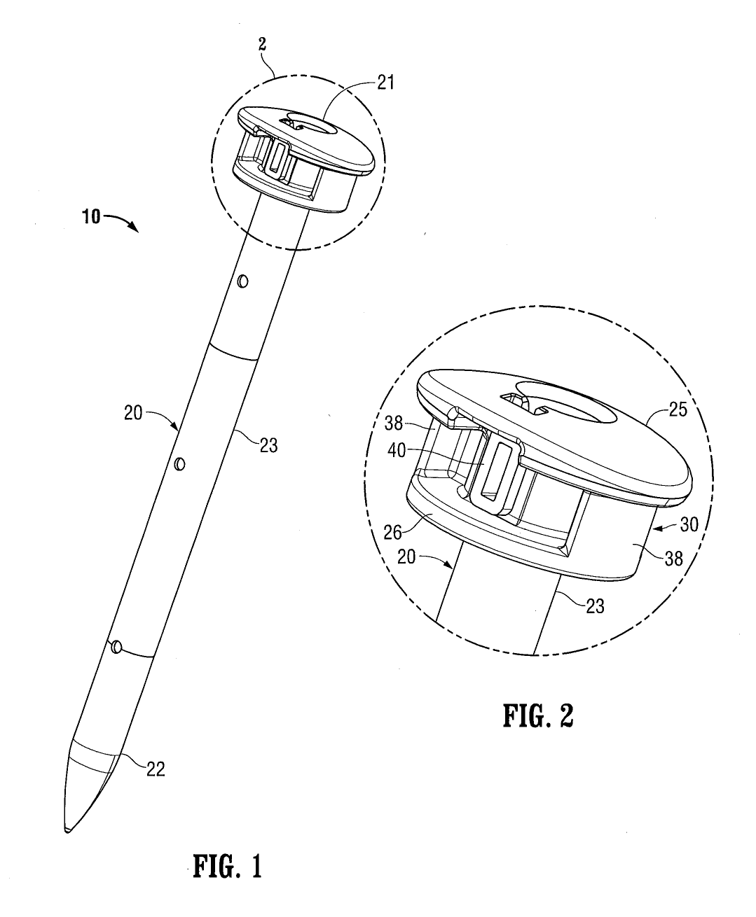

Referring now to the drawings, in which like reference numerals identify identical or substantially similar parts throughout the several views, FIG. 1 illustrates one exemplary embodiment of surgical device 10 which is in accordance with the present disclosure.

Device 10 includes elongate tubular member 20 having proximal end 21, distal end 22, and sidewall 23. Sidewall 23 defines bore 24 extending between proximal end 21 and distal end 22 as shown in FIG. 8. Sidewall 23 may include holes as shown in FIG. 1.

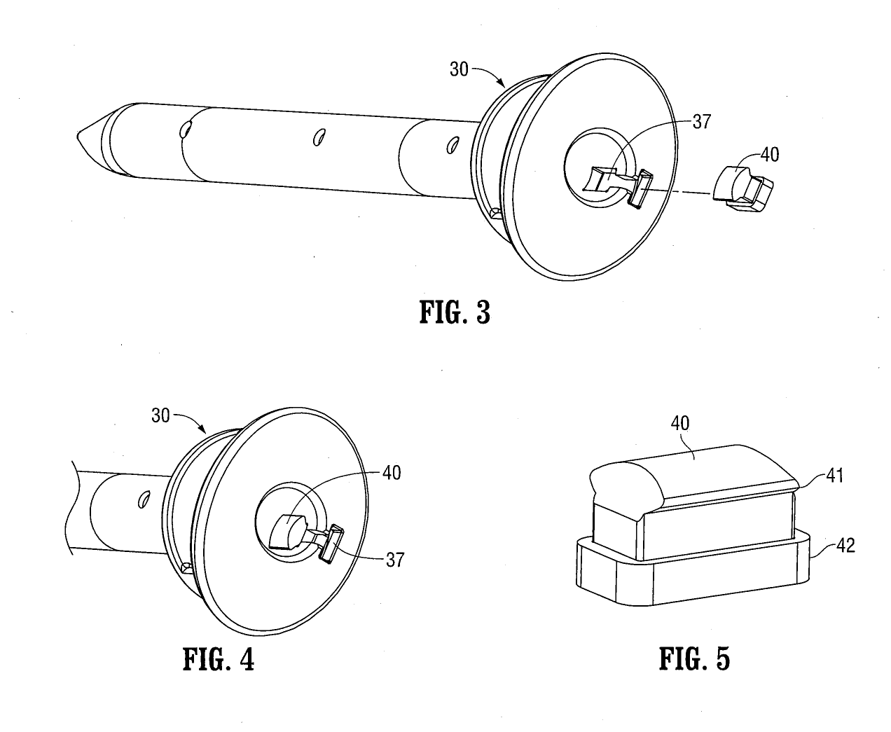

Now referring to FIG. 2, elongate tubular member 20 includes first and second flanges 25, 26 which extend radially from sidewall 23. First and second flanges 25, 26 may be generally cylindrical. First and second flanges 25, 26 may have the same diameter or different diameters with either first flange 25 having a diameter larger or smaller than second flange 26. First and second flanges 25, 26 may have the same thickness or different thicknesses with the thickness of first flange 25 being greater or less than the thickness of second flange 26. First and second flanges 25, 26 may have surfaces perpendicular to a longitudinal axis of the elongate tubular member 20 or have surfaces at angle other than perpendicular to the longitudinal axis of the elongate tubular member 20. Further, first and second flanges 25, 26 may also be parallel with respect to each other or be askew with respect to each other. The proximal and distal surfaces of first and second flanges 25, 26 may be parallel with respect to each other or be askew with respect to each other.

First and second flanges 25, 26 define housing 30 therebetween. Housing 30 may include instrument retention element 40 as shown in FIG. 2. Housing 30 may include wall 38 between first and second flanges 25, 26 as shown in FIGS. 12 and 13. Bore 24 may define through slot 39 in wall 38.

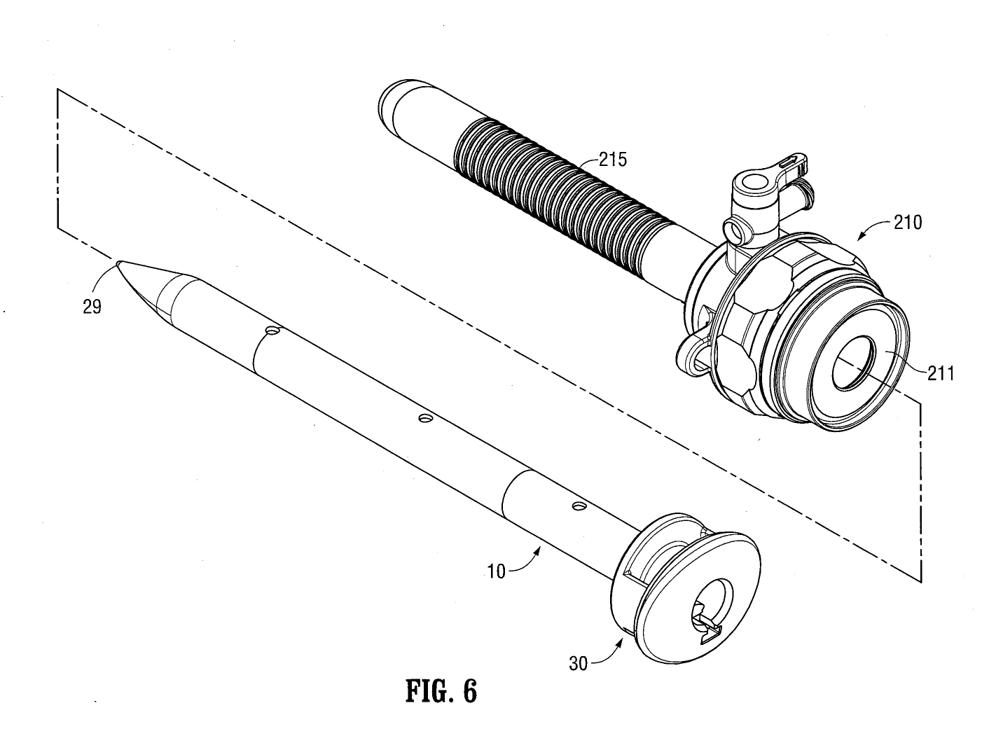

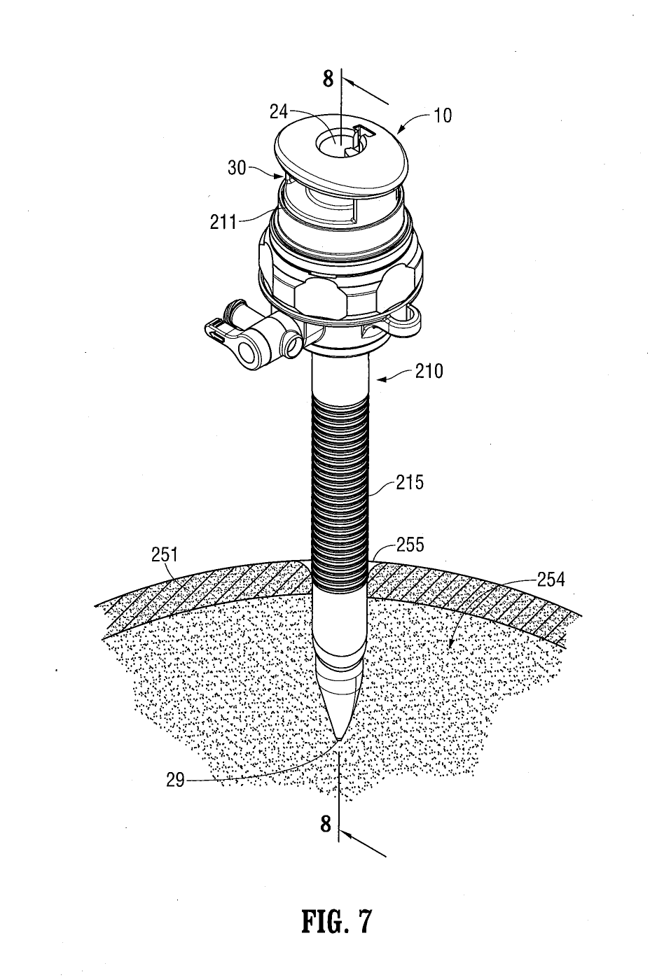

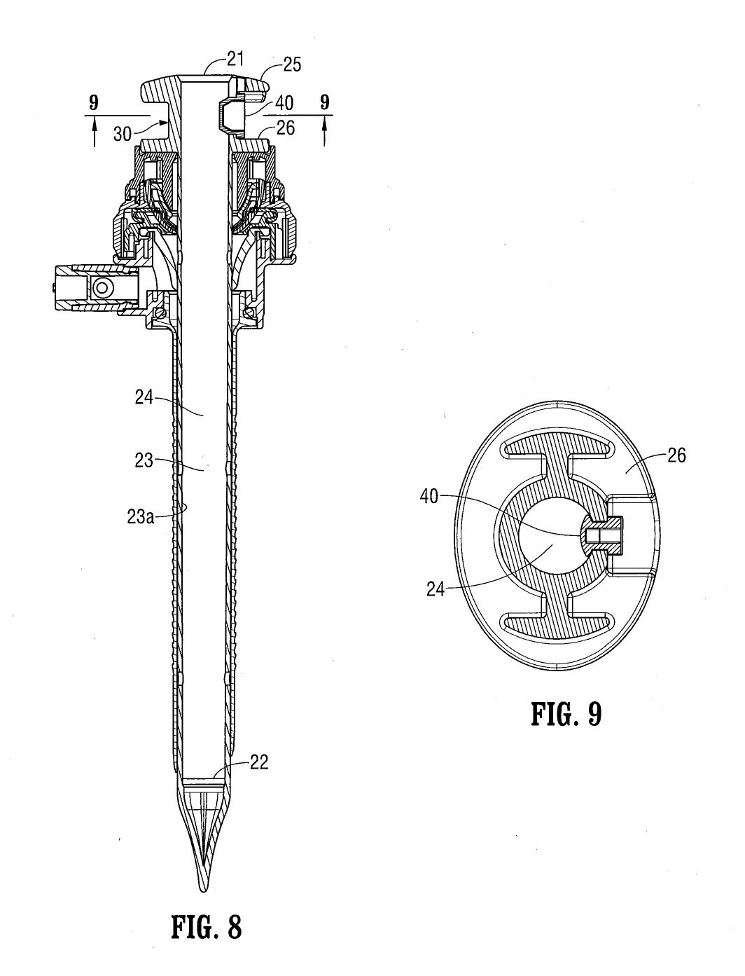

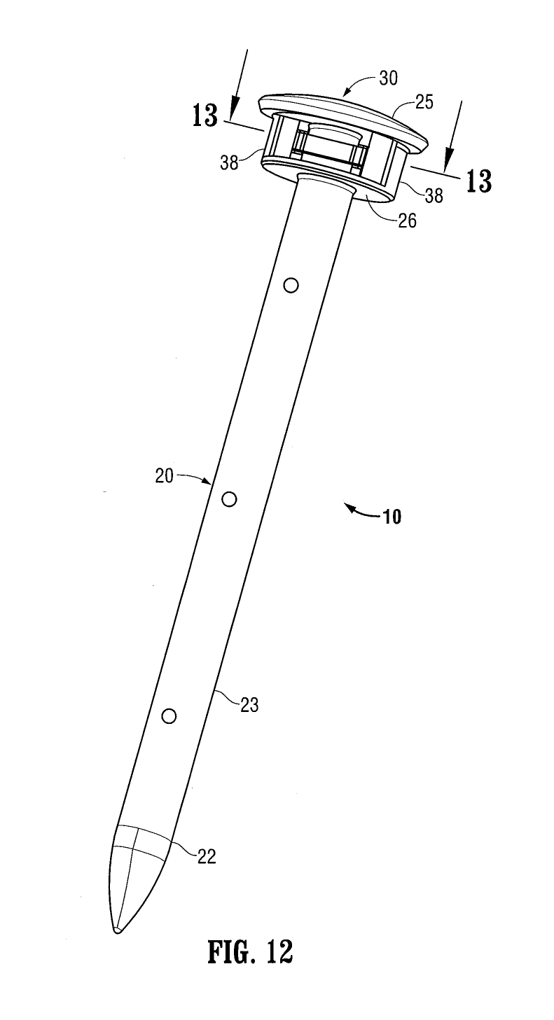

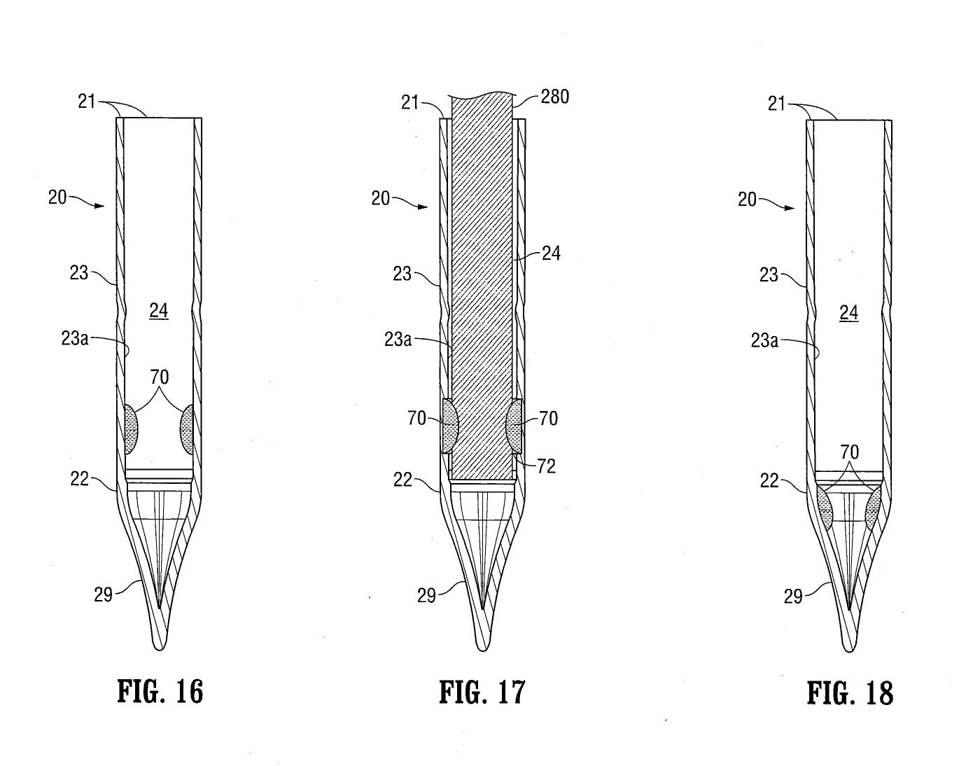

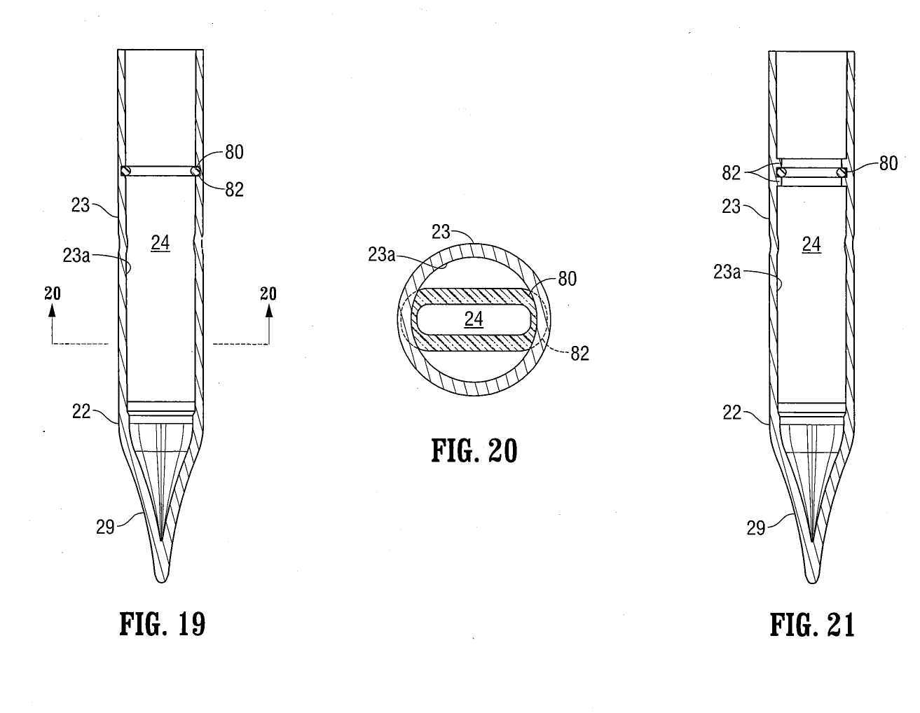

Now referring to the embodiment shown in FIGS. 3-5, instrument retention element 40 partially obstructs bore 24. Instrument retention element 40 is disposed within slot 37 in housing 30. Slot 37 extends through sidewall 23. Instrument retention element 40 is configured to selectively engage instrument 280 as shown in FIGS. 10 and 11. Instrument retention element 40 may include inner flange 41 and outer flange 42 configured to retain instrument retention element 40 within slot 37 as shown in FIG. 5. Instrument 280 may allow a clinician to directly or indirectly see within body cavity 254 through surgical device 10 as shown in FIG. 7. Instrument 280 may be a scope. Instrument 280 may also have a plurality of diameters. Instrument retention element 40 may also be configured to restrict the longitudinal and radial movement of the instrument 280.

In a particular embodiment of device 10, instrument retention element 40 is made from a compliant material. In this embodiment, instrument retention element 40 partially deforms when engaging instrument 280 as shown in FIGS. 10 and 11. The surface characteristics of instrument retention element 40 may be configured such that the friction between the surface of instrument retention element 40 and instrument 280 restricts the longitudinal and radial movement of instrument 280.

In another embodiment of device 10 shown in FIGS. 12-15, instrument retention element 60 is made from a rigid material. Instrument retention element 60 may include one or two legs 61 and backspan 62. Backspan 62 is connected to leg 61. When instrument retention element 60 has two legs 61, backspan 62 connects legs 61 together. Leg 61 may include locking mechanism 65. Locking mechanism 65 may be a tab 66 configured to be received by housing 30. Tab 66 may be configured to act like a clip with detent 67 engaging wall 38 of housing 30.

Referring now to FIGS. 14 and 15, instrument retention element 60 may act like a leaf spring when engaging instrument 280. When instrument 280 is inserted into bore 24 past instrument retention element 60, instrument 280 moves backspan 62 in the direction of the arrow shown in FIG. 15. In turn, instrument retention element 60 exerts a force on instrument 280. This force restricts the longitudinal and radial movement of instrument 280 with respect to device 10.

In yet another embodiment of device 10 illustrated in FIGS. 16-18, instrument retention element 70 is located near distal end 22 of device 10. Instrument retention element 70 may include a single structure or a plurality of structures positioned radially for at least partially obstructing bore 24. Alternatively, instrument retention element 70 may be a ring. Instrument retention element 70 may be constructed of a compliant material which partially deforms when receiving instrument 280. Instrument retention element 70 may restrict the longitudinal and radial movement of instrument 280. Instrument retention element 70 may be attached to inner surface 23a of sidewall 23 or located in recess 52 in sidewall 23. Instrument retention element 70 may also be located in tip 29 which is positioned at distal end 22 of elongate tubular member 20 as shown in FIG. 18. Instrument 280 may include structure 281 configured to engage instrument retention element 70 when structure 281 passes instrument retention element 70 as shown in FIG. 17. Structure 281 may be a recess, a detent, a rib, or any known means for engaging instrument retention element 70.

In still another embodiment of device 10 illustrated in FIGS. 19-21, instrument retention element 80 is located between proximal and distal ends 21, 22. In this embodiment device 10 may include housing 30 near proximal end 21. Instrument retention element 80 is be made from an elastomeric material which expands when receiving instrument 280. The interference between instrument 280 and instrument retention element 80 restricts the longitudinal and radial movement of instrument 280. Instrument 280 may include a structure 281 configured to engage instrument retention element 80 when structure 281 passes instrument retention element 80. Structure 281 may be a recess, a detent, a rib, or any known means for engaging instrument retention element 80. The elastomeric material of instrument retention element 80 may form an O-ring.

This embodiment may include retainer 82 in interior surface 23a of sidewall 23 configured to retain instrument retention element 80 in a fixed longitudinal position within bore 24. Retainer 82 may be a depression as shown in FIGS. 19 and 20. The depression may form an annular groove (not shown). Retainer 82 may be parallel ribs as shown in FIG. 21. There may be a plurality of parallel ribs displaced around the circumference of interior surface 23 where each parallel rib is across from an adjacent rib or each parallel rib may be across from a space between adjacent ribs. The parallel ribs may also form at least one complete ring around the circumference of interior surface 23.

In still yet another embodiment of device 10 illustrated in FIGS. 22 and 23, instrument retention element 90 is located at proximal end 21 of device 10. Instrument retention element 90 includes flange 91 expanding radially from elongate tubular member 20. Flange 91 partially obstructs bore 24 and includes longitudinal slot 82. Flange 91 may include a plurality of slots 82. When instrument retention element 90 receives instrument 280, slot 82 expands. The interference between flange 91 and instrument 280 restricts the longitudinal and radial movement of instrument 280. Instrument 280 may include a structure 281 configured to engage flange 91 when structure 281 passes flange 91. Structure 281 may be a recess, a detent, a rib, or any known means for engaging flange 91.

In another embodiment, the present disclosure may relate to a surgical device in which the instrument retention element resides and is supported on the outer surface of the obturator, but extends into the longitudinal channel of the obturator through bores defined by a sidewall of the obturator. An example embodiment of such an arrangement is shown in, e.g., FIGS. 24 and 25. As shown therein, an obturator includes a sidewall 23 having an arcuate outer surface 23b. In addition, the sidewall 23 has an arcuate inner surface 23a that defines a central longitudinal channel, e.g., bore 24, into which an instrument may be inserted. The sidewall 23 further defines at least one bore 29 extending between the arcuate inner and outer surfaces 23a, 23b of the sidewall 23. The instrument retention element 80 engages the arcuate outer surface 23b of the obturator such that the instrument retention element 80 extends radially into the bore 29. At least a portion of the instrument retention element 80 extends radially inward of the arcuate inner surface 23a so as to engage an instrument inserted into the central longitudinal channel of the obturator.

Advantageously, and as set forth above in connection with other embodiments described herein, the instrument retention element 80 may be made from an elastomeric material. The instrument retention element 80 may be an o-ring. It should also be noted that the instrument retention element 80 may be located on either the elongate tubular member or the handle located at the proximal end of the obturator.

The embodiment shown in FIGS. 24 and 25 may have particular advantages. For example, use of such an arrangement during manufacture may significantly simplify the assembly process as well as improve the safety and effectiveness of the device. For example, rather than the instrument retention element, e.g., o-ring, being inserted into the interior of the obturator (which could be quite difficult to accomplish in view of the narrow channel into which it must be inserted), the instrument retention element may be mounted on the outside surface of the obturator during the assembly processed and then moved, e.g., rolled, into its appropriate longitudinal position (where the bores 29 are located). Furthermore, by supporting the instrument retention element on the outer surface of the obturator, there is far less likelihood of the instrument retention element becoming dislodged from the obturator than if the instrument retention element is supported by the interior surface of the obturator alone. Dislodgement of the instrument retention element during surgery could pose a serious concern if it was to fall, or be pushed, into a body cavity by the obturator.

In order to use the surgical device shown in FIGS. 24 and 25, a user may insert an instrument, e.g., an endoscope, into the central longitudinal channel of the obturator such that the instrument retention element 80 engages the instrument and restricts longitudinal movement of the instrument relative to the obturator. The user may then use the obturator to engage, e.g., penetrate, tissue during a surgical procedure. In embodiments wherein the obturator is formed of a clear material, the endoscope may provide a visual image of the tissue being penetrated.

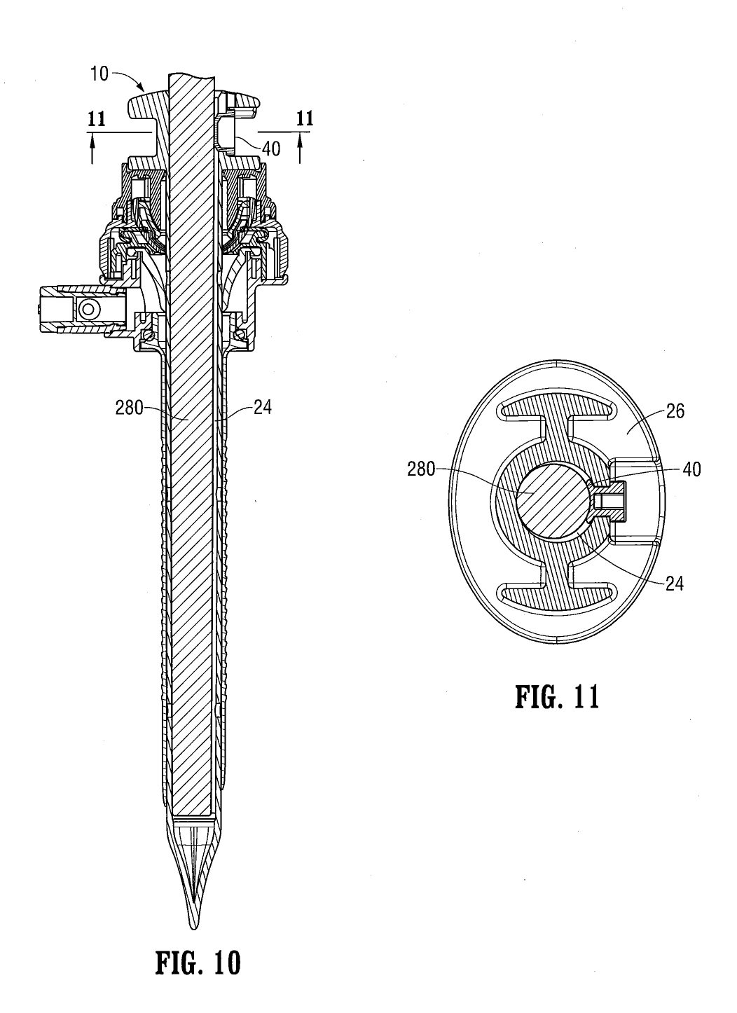

Referring now to FIGS. 6 and 7, a method for using surgical device 10 is disclosed. First, incision 255 is made in tissue layer 251. Incision 255 may be created by tip 29 of surgical device 10, by any other known methods such as a scalpel, or may be a naturally occurring orifice. Cannula 210 is then placed within incision 255 to provide access to a surgical working site. Fixing structure 215 on cannula 210 is configured to maintain the longitudinal and angular position of cannula 210 within incision 255 in relation to body cavity 254.

Once cannula 210 is secured in a desired position, surgical device 10 is then inserted and secured to cannula 210. Alternatively, if surgical device 10 was used to create incision 255 or surgical device 10 is inserted in a natural orifice, surgical device 10 may be is inserted in cannula 210 before incision 255 is created. Once inserted cannula 210 is in the desired position, surgical device 10 is inserted into body cavity 254 until housing 30 mates with the proximal end 211 of cannula 210. Housing 30 may be configured to secure surgical device 10 to cannula 210.

When surgical device 10 is in the desired position, the clinician inserts instrument 280 into bore 24. When instrument 280 is inserted into bore 24, instrument 280 is engaged by an instrument retention element (any of the disclosed instrument retention elements 40, 60, 70, 80, or 90 may be used). The instrument retention element may be configured to allow the clinician to insert instrument 280 to a desired position within bore 24 while maintaining instrument 280 in the desired position once the clinician releases instrument 280.

Once in the desired position, the clinician is able to directly view body cavity 254 through instrument 280 or indirectly view body cavity 254 on a monitor (not shown) receiving a signal from instrument 280.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope and spirit of the claims appended hereto.

What is claimed:

1. A surgical device, comprising:
an obturator including a sidewall having an arcuate inner surface that defines a central longitudinal channel into which an instrument may be inserted; and
an instrument retention element engaging the sidewall of the obturator such that at least a portion of the instrument retention element extends radially inward of the arcuate inner surface so as to engage an instrument inserted into the central longitudinal channel of the obturator.

2. The surgical device of claim 1, wherein the instrument retention element is configured to engage an endoscope.

3. The surgical device of claim 1, wherein the instrument retention element is configured to engage instruments of varying diameters.

4. The surgical device of claim 1, wherein the instrument retention element is configured to restrict the longitudinal movement of the instrument relative to the obturator.

5. The surgical device of claim 1, wherein at least a portion of the instrument retention element is made from an elastomeric material.

6. The surgical device of claim 1, wherein the instrument retention element is an o-ring.

7. The surgical device of claim 1, wherein the arcuate inner surface has a retainer configured to retain the instrument retention element.

8. The surgical device of claim 7, wherein the retainer includes at least one of a depression, an annular groove, a rib, or a ring.

9. The surgical device of claim 1, wherein the obturator includes an elongate tubular member which forms a portion of the sidewall, the instrument retention element being located on the elongate tubular member.

10. The surgical device of claim 1, wherein the obturator includes a handle which forms a portion of the sidewall, the instrument retention element being located on the handle.

11. A surgical device, comprising:
an obturator including a sidewall having an arcuate outer surface, the sidewall having an arcuate inner surface that defines a central longitudinal channel into which an instrument may be inserted, the sidewall further defining at least one bore extending between the arcuate inner and outer surfaces of the sidewall; and
an instrument retention element engaging the arcuate outer surface of the sidewall of the obturator such that the instrument retention element extends radially into the bore, at least a portion of the instrument retention element extending radially inward of the arcuate inner surface so as to engage an instrument inserted into the central longitudinal channel of the obturator.

12. The surgical device of claim 11, wherein the instrument retention element is configured to engage an endoscope.

13. The surgical device of claim 11, wherein the instrument retention element is configured to engage instruments having varying diameters.

14. The surgical device of claim 11, wherein the instrument retention element is further configured to restrict the longitudinal movement of the instrument relative to the obturator.

15. The surgical device of claim 11, wherein at least a portion of the instrument retention element is made from an elastomeric material.

16. The surgical device of claim 11, wherein the instrument retention element is an o-ring.

17. The surgical device of claim 11, wherein the obturator includes an elongate tubular member which forms a portion of the sidewall, the instrument retention element being located on the elongate tubular member.

18. The surgical device of claim 11, wherein the obturator includes a handle which forms a portion of the sidewall, the instrument retention element being located on the handle.

19. A method of using a surgical device, comprising:
providing an obturator including a sidewall having an arcuate outer surface, the sidewall having an arcuate inner surface that defines a central longitudinal channel, the sidewall further defining at least one bore extending between the arcuate inner and outer surfaces of the sidewall, an instrument retention element engaging the arcuate outer surface of the obturator such that the instrument retention element extends radially into the bore, at least a portion of the instrument retention element extending radially inward of the arcuate inner surface;
inserting an instrument into the central longitudinal channel of the obturator such that the instrument retention element engages the instrument and restricts longitudinal movement of the instrument relative to the obturator; and
using the obturator to engage tissue during a surgical procedure.

20. The method of claim 19, wherein at least a portion of the obturator is formed of a clear material and wherein the instrument is an endoscope, and wherein the step of using the obturator includes penetrating tissue with the obturator while the endoscope provides a visual image of the tissue being penetrated.
